(19) United States
(12) Patent Application Publication
(10) Pub. No.: US 2021/0169335 A1
(43) Pub. Date: Jun. 10, 2021

(54) HEALTH CONDITION MANAGEMENT SYSTEM, METHOD FOR CONTROLLING HEALTH CONDITION MANAGEMENT SYSTEM, AND PROGRAM

(71) Applicants: ShinSei Co., Ltd., Kyoto (JP); PIEZOTECH JAPAN Ltd., Kyoto (JP)

(72) Inventors: Ryutaro ASHIDA, Kyoto (JP); Elia MARIN, Kyoto (JP)

(21) Appl. No.: 16/768,621

(22) PCT Filed: Nov. 30, 2017
(86) PCT No.: PCT/JP2017/043210

§ 371 Date: May 29, 2020

(51) Int. Cl. A61B 5/00; G16H 50/30; G16H 40/20; G16H 10/60; G16H 50/20

(52) U.S. Cl. CPC A61B 5/0075; A61B 5/0088; A61B 5/7282; A61B 2576/02; G16H 40/20; G16H 10/60; G16H 50/20; G16H 50/30

(57) ABSTRACT

This health condition management system includes a measurement terminal including a Raman spectroscopy device and a mobile terminal; a server; and a hospital terminal. The mobile terminal transmits measurement data from Raman spectrometry to the server, and the server determines a tooth condition on the basis of a predetermined determination algorithm from the received measurement data, and transmits same to the mobile terminal and the hospital terminal. In addition, the server selects a dental clinic to visit on the basis of the determined condition and registration information, and transmits same to the mobile terminal. Appointment/reception assistance is also performed between the mobile terminal and the hospital terminal, with the ability to determine an appointment date and time.

### TECHNICAL FIELD

The invention relates to a health condition management system that measures and manages a health condition of a subject by applying a spectroscopic technique, a method for controlling the health condition management system, and a program.

### BACKGROUND ART

Conventionally, a method for analyzing a physical property value using a spectroscopic technique has been known. Regarding this technique, for example, there is known a method for analyzing a physical property value of a polymer material, in which the physical property value of the polymer material to be analyzed of the same kind as the specific polymer material is analyzed with reference to a calibration curve representing a relationship between a Raman spectrum value and a physical property value prepared in advance for the specific polymer material. The method includes a step for measuring a Raman spectrum of the polymer material to be analyzed, a step for calculating the same kind of spectrum value in the reference from this Raman spectrum, and a step for analyzing the physical property value corresponding to the spectrum value using the reference (see Patent Literature 1).

On the other hand, as a system for health management of a subject, there is known a system which includes a spectral analyzer that irradiates food with detection light and receives a reflected light reflected from the food, a component analysis unit that performs component analysis of the food based on the reflected light, a mass detection unit that detects the mass of the food, an identification unit that identifies each acquirer by acquiring individual information that identifies each of multiple acquirers, and a storage unit that individually stores individual information of each acquirer and the acquired result of the component analysis of the food acquired by each acquirer (see Patent Literature 2). This is a health management system that manages the component analysis result of the inspection target acquired by a plurality of acquirers on an acquirer basis.

In addition, there is known a system which includes a sensor housing installed in the temple of the subject. The sensor housing includes a biological optical measurement device for measuring a hemoglobin signal change accompanying a functional change of a salivary line in the vicinity of the temple of the subject, and a chewing motion detection device and a sensor control device for detecting the temple movements associated with mastication of the subject. The biological light measuring device and the chewing motion detection device are operated simultaneously, and the measurement result is sent to an operating company system. The measurement result is displayed on a display device together with recommendation information acquired from this operating company system (see Patent Literature 3).

It cannot be said that the above-mentioned conventional technique is able to directly measure the health condition of the subject to be managed more easily and accurately. Further, the measurement data of the health condition is not effectively used to efficiently maintain or improve the health condition of the subject by a specialized institution such as a medical institution.

### CITATION LIST

### Patent Literature

### Patent Literature 1: JP 4384571 B1

### Patent Literature 2: JP 2014-163872 A

### Patent Literature 3: JP 2012-208735 A

### SUMMARY OF INVENTION

### Technical Problem

The invention has been invented in view of the above situation, and an object thereof is to provide a technology that the subject to be managed can directly measure its health condition more easily and accurately, and further a specialized institution can maintain or improve the health condition of the subject more efficiently using the measurement data of the health condition.

### Solution to Problem

According to the invention for solving the above problems, there is provided a health condition management system which includes a spectroscopic measurement device that irradiates a part of a subject's body with light and detects a scattered light or a reflected light from the subject's body to perform spectroscopic measurement, a subject terminal to which an output signal of the spectroscopic measurement device is input, an information processing device that is capable of communicating with the subject terminal, and a management institution terminal that is provided in a management institution for managing a health condition of the subject and capable of communicating with the subject terminal and the information processing device. The subject terminal includes an input unit to which a measurement result by the spectroscopic measurement device is input, and a transmission unit that transmits the measurement result input to the input unit to the information processing device. The information processing device includes a measurement information receiving unit that receives the measurement result transmitted from the transmission unit of the subject terminal, a determination unit that determines a health condition of the subject based on the measurement result received by the measurement information receiving unit and a predetermined algorithm, and a determination information transmitting unit that transmits determination information that is information of a result determined by the determination unit. The management institution terminal includes a determination information receiving unit that receives the determination information transmitted by the determination information transmitting unit of the information processing device. The information processing device further includes a schedule planning unit that determines a management plan of a health condition of the subject based on the determination information, and a schedule information transmitting unit that transmits a schedule information that is information of a management schedule of a health condition of the subject determined by the schedule planning unit to the subject terminal and/or the management institution terminal. The subject terminal includes a second determination information receiving unit that receives the determination information transmitted by the determination information transmitting unit of the information processing device, and a schedule information receiving unit that receives the schedule information transmitted by the schedule information transmitting unit of the information processing device. The management institution terminal includes a second schedule information receiving unit that receives the schedule information transmitted by the schedule information transmitting unit of the information processing device.

According to this, the subject can automatically acquire the determination result of the health condition by performing the spectroscopic measurement on a part of the body with the spectroscopic measurement device and transmitting the measurement result to the information processing device. Further, it is possible to automatically obtain schedule information about the management schedule based on the determination result of the health condition. Therefore, the subject can promptly have the specialized institution maintain or improve the health condition. Here, being based on the determination result of the health condition includes that the determination result indicates that the condition requires management.

Here, the spectroscopic measurement may be one in which the body of the subject is simply irradiated with light and the spectroscopic characteristics of the reflected light are examined, or one that the peak of scattered light excited depending on the molecular structure in a part of the subject is detected. In addition, examples of the health condition here include, but are not limited to, the tooth condition of the subject, the skin age, the BMI and fat percentage that can be estimated from the skin condition, the osteoporosis condition, the bone necrosis condition, and the like.

In addition, in the above, examples of the management institution include hospitals, various clinics, dental clinics, specialized institutions such as counselors, and nursing facilities such as nursing homes. In addition, the management schedule of the health condition includes a schedule of going to the management institution, a schedule of an interview with an expert of the management institution, and the like.

Further, in the invention, the subject terminal may include an additional information transmitting unit that transmits additional information regarding a health condition of the subject to the management institution terminal. The management institution terminal may further include an additional information receiving unit that receives the additional information transmitted from the additional information transmitting unit of the subject terminal.

Here, the additional information may be a description of a symptom related to the measurement result measured by the spectroscopic measurement device, an image, or the like. Alternatively, it may be a related consultation message or the like. As a result, more detailed and flexible information can be exchanged between the management institution and the subject, and the subject's health condition can be maintained or managed more reliably.

Further, in the invention, the management institution terminal may further include a guide information transmitting unit that transmits guide information for guiding the subject to be managed by the management institution based on the determination information. The subject terminal may further include a guide information receiving unit that receives the guide information transmitted by the guide information transmitting unit.

Here, the guide information may be, for example, a guide message of a visit from the hospital to the subject. In addition, it may be an instruction message from the hospital or the like to the subject about the medication. According to this, it is possible to provide more detailed guidance and instruction from the management institution to the subject, and it is possible to more reliably maintain or manage the subject's health condition.

Further, in the invention, the subject terminal may further include a personal information transmitting unit that transmits personal information of the subject to the information processing device. The management institution terminal may further include an institution information transmitting unit that transmits institution information of the management institution to the information processing device. The information processing device may further include a personal information receiving unit that receives the personal information, an institution information receiving unit that receives the institution information, a personal information storage unit that stores one or more pieces of the personal information, and an institution information storage unit that stores one or more pieces of the institution information. The schedule planning unit may determine a management schedule of the health condition of the subject based on the personal information of the subject stored in the personal information storage unit and the institution information of the management institution stored in the institution information storage unit.

That is, in the health condition management system according to the invention, the subject's personal information is transmitted from the subject terminal to the information processing device and registered. Also, the institution information of the management institution is transmitted from the management institution terminal to the information processing device and registered. In the information processing device, these pieces of information are made into a database. Then, in the information processing device, the management schedule of the health condition of the subject is determined based on the personal information of the subject and the institution information of the management institution, which are stored in the database, in addition to the above determination result. For example, the management schedule may be set such that a specialized hospital related to the determination result will visit a hospital in the same area as the subject's residence.

Also, in the invention, the institution information may include information on name, location, and contact information of the management institution. The personal information may include information about the subject's residence. The schedule planning unit may select a management institution whose residence is closer to the subject's residence. The schedule information may include at least one of name, address, and contact information of the selected management institution. According to this, it is possible to more easily determine the management schedule of the health condition of the subject.

Further, in the invention, the subject terminal further may include a reservation information transmitting unit that transmits, to the information processing device and/or the management institution terminal, reservation information including a desired reservation date, which is included in the schedule information and desired to be reserved in the management institution selected by the schedule planning unit. The information processing device and/or the management institution terminal may further include a reservation information receiving unit that receives the reservation information.

Thereby, the subject can more easily confirm the reservation to the management institution by the schedule information transmitted from the information processing device. This makes it possible to promote the maintenance or improvement of the health condition of the subject more quickly.

Further, in the invention, the spectroscopic measurement device may be a Raman spectroscopic device that spectroscopically measures Raman scattered light from the subject using a Raman spectroscopic technique. According to this, spectroscopic measurement having a high correlation can be performed due to the molecular structure in a part of the body of the subject, and the health condition can be determined with higher accuracy.

Further, in the invention, the information processing device may include a determination reference storage unit that stores a relationship between a calculated value obtained by adding a predetermined calculation to at least one of a Raman scattered light intensity at a predetermined frequency, a peak width, and a peak position and a health condition of the subject. The determination unit refers the measurement result received by the measurement information receiving unit to the relationship stored in the determination reference storage unit, and determines the health condition of the subject based on an algorithm for deriving the health condition of the subject which corresponds to the measurement result.

Here, in the Raman scattering spectroscopic measurement, it has been found that at least one of the peak position, the peak intensity, and the peak width at a specific wave number shift is measured, and the calculated value obtained by adding a predetermined calculation to these has the high correlation with the health condition of the biological sample. In the invention, the health condition of the subject is determined using the relationship between the calculated value and the health condition of the biological sample, which has the high correlation. According to this, it is possible to very accurately determine the health condition of the subject.

Further, in the invention, a part of the subject's body may be a subject's teeth. The health condition may be a tooth condition. The management institution may be a dental clinic.

Here, it has been found that the Raman scattering spectroscopic measurement enables very accurate tooth diagnosis. Therefore, according to the invention, for example, it is possible to determine the tooth condition very accurately at home. In addition, it is possible to efficiently connect with many dental clinics existing in the area and efficiently promote the prevention and treatment of diseases such as dental caries.

Further, the invention may provide a method for controlling a health condition management system that includes a spectroscopic measurement device that irradiates a part of a subject's body with light and detects a scattered light or a reflected light from the subject's body to perform spectroscopic measurement, a subject terminal to which an output signal of the spectroscopic measurement device is input, an information processing device that is capable of communicating with the subject terminal, and a management institution terminal that is provided in a management institution for managing a health condition of the subject and capable of communicating with the subject terminal and the information processing device. The method includes a spectroscopic measurement step for spectroscopically measuring a part of the subject's body by the spectroscopic measurement device, a measurement result transmitting step for transmitting a measurement result by the spectroscopic measurement step from the subject terminal to the information processing device, a determination step for determining the health condition of the subject in the information processing device based on the measurement result and a predetermined algorithm, a determination information transmitting step for transmitting determination information, which is information of a result determined in the determination step, from the information processing device to the subject terminal, a schedule planning step for determining a management schedule of the health condition of the subject based on the determination information, and a schedule information transmitting step for transmitting schedule information, which is information on the management schedule of the health condition of the subject determined in the schedule planning step, from the information processing device to the subject terminal.

Further, in the invention, the method for controlling the health condition management system may further include an additional information transmitting step for transmitting additional information related to the health condition of the subject from the subject terminal to the management institution terminal.

Further, in the invention, the method for controlling the health condition management system may further include a determination information transmitting step for transmitting determination information, which is information of a result determined in the determination step, from the information processing device to the management institution terminal, and a guide information transmitting step for transmitting guide information for guiding the subject to be managed by the management institution from the management institution terminal to the subject terminal based on the determination information.

Further, in the invention, the method for controlling a health condition management system may include a personal information transmitting step for transmitting personal information of the subject from the subject terminal to the information processing device, an institution information transmitting step for transmitting institution information of the management institution to the information processing device from the management institution terminal, a personal information storing step for receiving and storing one or more pieces of the personal information in the information processing device to form a database, and an institution information storing step for receiving and storing one or more pieces of the institution information in the management institution terminal to form a database. In the schedule planning step, a management schedule of the health condition of the subject may be determined based on the determination information, the personal information of the subject stored in the database in the personal information storing step, and the institution information of the management institution stored in the database in the institution information storing step.

Further, in the method for controlling the health condition management system according to the invention, the institution information may include information on name, location, and contact information of the management institution. The personal information may include information about the subject's residence. In the schedule planning step, a management institution whose location may be closer to the subject's residence is selected, and the schedule information may include at least one of name, location, and contact information of the selected management institution.

Further, in the invention, the method for controlling the health condition management system may further include a reservation information transmitting step for transmitting, from the subject terminal to the information processing device and/or the management institution terminal, reservation information including a desired reservation date, which is included in the schedule information and desired to be reserved in the management institution selected in the schedule planning step.

Further, the invention may be a method for controlling the health condition management system, in which Raman spectroscopic measurement may be performed to spectroscopically measure Raman scattered light from the subject using a Raman spectroscopic technique in the spectroscopic measurement step.

Further, the invention, the method for controlling the health condition management system may further include a determination reference forming step for storing a determination reference for storing a relationship between a calculated value obtained by adding a predetermined calculation to at least one of a Raman scattered light intensity at a predetermined frequency, a peak width, and a peak position and a health condition of the subject. The predetermined algorithm may be an algorithm for referring the measurement result transmitted to the information processing device in the measurement result transmitting step to the relationship stored in the determination reference, and deriving the health condition of the subject which is corresponding to the measurement result.

Further, in the method for controlling the health condition management system according to the invention, a part of the subject's body may be the subject's teeth. The health condition may be a tooth condition. The management institution may be a dental clinic.

Further, the invention may be a program for causing at least one of the subject terminal, the information processing device, and the management institution terminal to execute the method for controlling the health condition management system.

In the invention, means for solving the above problems can be used in combination as much as possible.

### Advantageous Effects of Invention

According to the invention, a subject to be managed can directly measure its health condition more easily and accurately, and further a specialized management institution can maintain or improve the health condition of the subject more efficiently using the measurement data of the health condition.

### BRIEF DESCRIPTION OF DRAWINGS

FIG. 1 is a diagram illustrating an outline of a health condition management system in an embodiment of the invention.

FIG. 2 is a schematic diagram of the configuration of a Raman spectroscopic device in an embodiment of the invention.

FIG. 3 is a diagram illustrating a hardware configuration of a health condition management system in an embodiment of the invention.

FIG. 4 is a diagram illustrating a functional configuration of the health condition management system in an embodiment of the invention.

FIG. 5 is a flowchart of a measurement routine in the embodiment of the invention.

FIG. 6 is a flowchart of a determination routine in the embodiment of the invention.

FIG. 7 is a flowchart of a schedule planning routine in the embodiment of the invention.

FIG. 8 is a flowchart of a reservation acceptance support routine in the embodiment of the invention.

FIG. 9 is a graph illustrating a wave number distribution of Raman scattered light intensity on a tooth surface in the embodiment of the invention.

FIG. 10 is a graph illustrating the relationship between a Qt value and the degree of tooth damage in the embodiment of the invention.

FIG. 11 is a second graph illustrating the wave number distribution of the Raman scattered light intensity on the tooth surface in the embodiment of the invention.

FIG. 12 is a graph illustrating the relationship between a calculated value RPF of the Raman scattered light intensity on a skin surface and the age of a skin donor in the embodiment of the invention.

FIG. 13 is a graph illustrating the relationship between the calculated value RLC/BMI of the Raman scattered light intensity on the skin surface and the age of the skin donor in the embodiment of the invention.

FIG. 14 is a graph illustrating the difference between a peak position and a peak intensity distribution of phenylalanine of the Raman scattered light intensity on a bone surface in the embodiment of the invention depending on the state of the bone.

FIG. 15 is a second graph illustrating the difference in the peak position and the peak intensity distribution of sphingomyelin of the Raman scattered light intensity on the bone surface according to the embodiment of the invention depending on the state of the bone.

### DESCRIPTION OF EMBODIMENTS

Embodiments of the invention will be described below with reference to the drawings. Further, the embodiments described below are merely examples for implementing the invention, and the invention is not limited to the specific configurations described below.

### EMBODIMENTS

### <Outline of Health Management System>

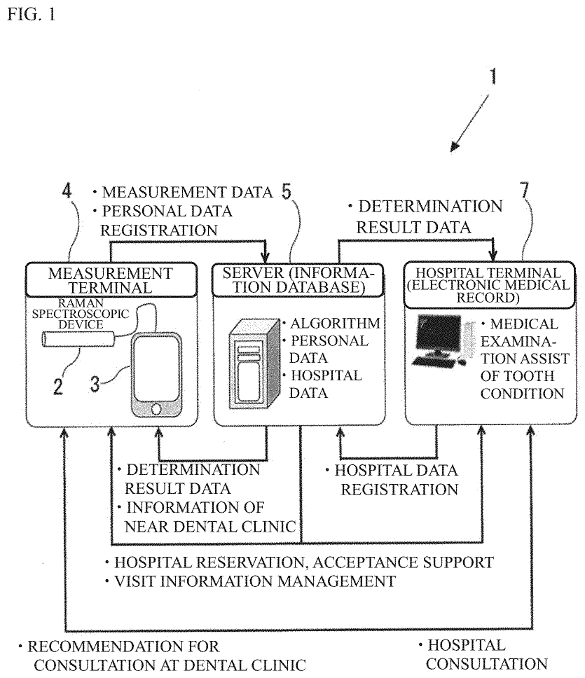

FIG. 1 is a diagram illustrating a health management system 1 in this embodiment. The health management system 1 has a measurement terminal 4 including a Raman spectroscopic device 2 and a mobile terminal 3, a server 5 that stores a database of various information, and a hospital terminal 7 that manages a patient by an electronic medical record or the like. In this embodiment, the health management system 1 corresponds to a health condition management system. The Raman spectroscopic device 2 corresponds to a spectroscopic measurement device. The mobile terminal 3 corresponds to the subject terminal. The server 5 corresponds to an information processing device. The hospital terminal 7 corresponds to the management institution terminal.

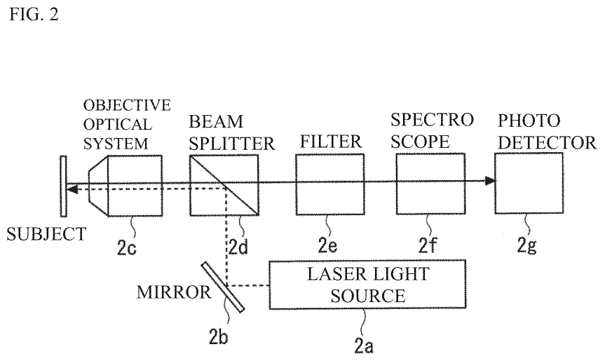

The Raman spectroscopic device 2 in the measurement terminal 4 has, for example, a configuration as illustrated in FIG. 2. As illustrated in FIG. 2, since the excitation light for exciting Raman scattering is required to be stable single-wavelength light, a laser light source 2a is used as the light source. The excitation light is reflected by a mirror 2b, then bent 90 degrees in a beam splitter 2d, and passes through an objective optical system 2c to be condensed on a subject.

Then, a part of the reflected light from the subject passes through the objective optical system 2c and the beam splitter 2d and enters a filter 2e. Here, a part of the reflected light from the subject is Rayleigh scattered light or the like. The reflected light also includes Raman scattered light due to Raman scattering in which the wave number of the excitation light is shifted according to the molecular structure of the subject. Then, this reflected light is made incident on the filter 2e and Rayleigh scattered light and the like are cut off. The reflected light that has passed through the filter 2e enters a spectroscope 2f. In the spectroscope 2f, the reflected light is separated into each wave number (wavelength) by a diffraction grating or the like. The separated Raman scattered light is irradiated to a photodetector 2g. The Raman scattered light can be detected by this photodetector 2g.

The description returns to FIG. 1. In the health management system 1 according to this embodiment, the mobile terminal 3 in the measurement terminal 4 downloads a dedicated application to control the Raman spectroscopic device 2 and acquire the output signal of the Raman spectroscopic device 2, and can transmit the output signal to the server 5. Further, in the mobile terminal 3, it is possible to register the personal data of the subject, for example, name, residence, sex, age, etc., in the server 5 using the dedicated application.

A determination algorithm (described later) for determining a tooth condition of the subject from the measurement data of the Raman spectroscopic device 2 is installed in the server 5. In addition, the personal data transmitted from the plurality of mobile terminals 3 is stored as a database. Further, as will be described later, the hospital terminal 7 transmits information on the dental clinic, for example, name, location, electronic mail address, etc., and the dental clinic information is stored in the server 5 as a database.

In addition, the server 5 transmits determination result data regarding the tooth condition determined by the determination algorithm to the mobile terminal 3 and the hospital terminal 7. Further, the server 5 has a selection algorithm which compares the personal data transmitted from the mobile terminal 3 with the database storing the hospital data of the dental clinic transmitted from the hospital terminal 7, and selects a dental clinic suitable for the subject. Then, the determination result data regarding the tooth condition determined by the determination algorithm and the dental clinic information selected by the selection algorithm are transmitted from the server 5 to the mobile terminal 3. The selection reference of the dental clinic by the selection algorithm may be the proximity of the location of the dental clinic to the subject's residence, or the specialty field or the possessed equipment corresponding to the tooth condition. Further, other factors may be used as a reference.

In addition, the server 5 and the mobile terminal 3 exchange information about the desired visit date of the subject, and the server 5 and the hospital terminal 7 exchange information about the reservation available date to make a hospital reservation and to support reception. Then, by exchanging the information of the actual visit between the mobile terminal 3 and the hospital terminal 7, it is possible to manage the visit information.

In addition, it is possible to transmit a consultation message regarding dental health management, including the tooth condition, from the mobile terminal 3 to the hospital terminal 7. It is possible to transmit a guide message from the hospital terminal 7 to the mobile terminal 3 to recommend a consultation to a dental clinic. Here, the process of transmitting the consultation message from the mobile terminal 3 to the hospital terminal 7 corresponds to an additional information transmitting step in this embodiment. Further, the process of transmitting the guide message recommending a consultation to a dental clinic from the hospital terminal 7 to the mobile terminal 3 corresponds to a guide information transmitting step in this embodiment.

In addition, the server 5 transmits the determination result data, which is information on the determination result of the tooth condition, to the hospital terminal 7. An electronic medical record about the subject is created and stored in the hospital terminal 7. Then, the determination result data transmitted from the server 5 can assist the medical examination when the subject actually visits a hospital.

Here, the mobile terminal 3 may be, for example, a smartphone. The mobile terminal 3 may be a tablet terminal, smart glass, wearable device, or the like. The server 5 is a computer connected to a network by an Internet line. The hospital terminal 7 is, for example, a personal computer connected to the network by an Internet line. Instead of the Internet N, a LAN (Local Area Network), a WAN (Wide Area Network), or other packet communication networks may be adopted.

### <Hardware Configuration>

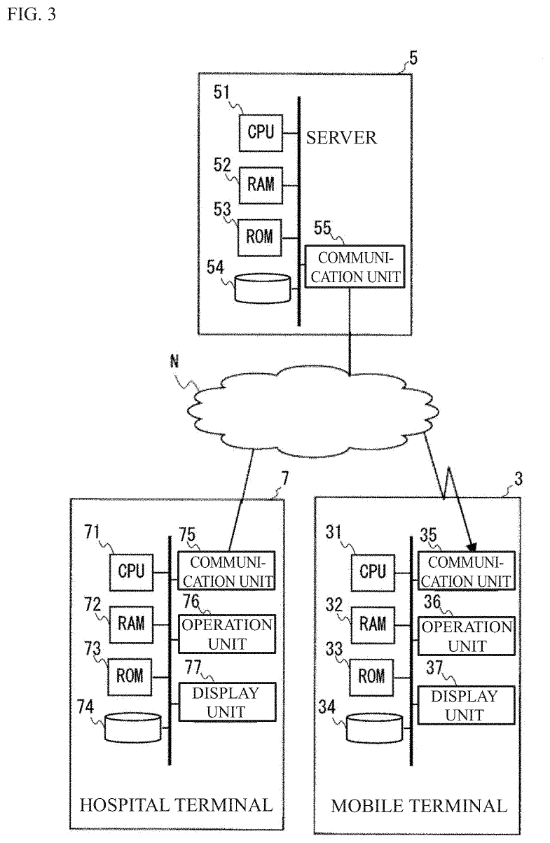

FIG. 3 is a diagram illustrating a hardware configuration of each device in this embodiment. FIG. 3 illustrates the hardware configuration of the mobile terminal 3, the server 5, and the hospital terminal 7.

The mobile terminal 3 is a terminal such as a smartphone which includes a CPU (Central Processing Unit) 31, a RAM (Random Access Memory) 32, a ROM (Read Only Memory) 33, an auxiliary storage device 34 such as SSD (Solid State Drive), a communication unit 35 connected to the Internet N, an operation unit 36 such as a touch panel, and a display unit 37 such as a display. The communication unit 35 is, for example, a NIC (Network interface controller) connected to the Internet N via an access point or the like of a wireless LAN, and may also be connected to the Internet N via a mobile phone communication network such as LTE (Long Term Evolution).

The CPU 31 is a central processing unit, and controls the RAM 32, the auxiliary storage device 34, and the like by processing commands and data expanded in the RAM 32 and the like. The RAM 32 is a main storage device and is controlled by the CPU 31, and various commands and data are written and read. The auxiliary storage device 34 is a non-volatile storage device and stores various programs, data required to be permanently saved, and the like.

The server 5 is a computer including a CPU 51, a RAM 52, a ROM 53, an auxiliary storage device 54 such as an HDD (Hard Disk Drive), and a communication unit 55 connected to the Internet N via a gateway or the like. Although the server 5 is illustrated as one computer in FIG. 3, the server 5 may be configured by, for example, a plurality of computers connected by a network.

The hospital terminal 7 is a computer which includes a CPU 71, a RAM 72, a ROM 73, an auxiliary storage device 74 such as an HDD, a communication unit 75 connected to the Internet N via a gateway, an operation unit 76 such as a keyboard and a mouse, and a display unit 77 such as a display. Although not illustrated, the hospital terminal 7 is connected to a printer that prints.

### <Functional Configuration>

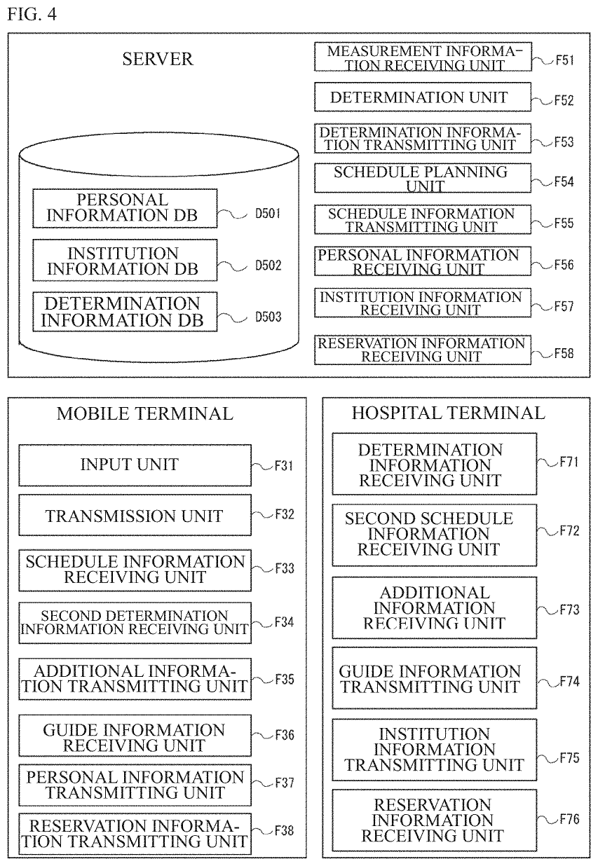

FIG. 4 is a diagram illustrating a functional configuration of each device according to this embodiment. FIG. 4 illustrates main functions of the mobile terminal 3, the server 5, and the hospital terminal 7.

### (Functions of Mobile Terminal 3)

In the mobile terminal 3, a program such as a health management application stored in the auxiliary storage device 34 is read into the RAM 32 and executed by the CPU 31. Thereby, the mobile terminal 3 functions as a computer which includes an input unit F31, a transmission unit F32, a schedule information receiving unit F33, a second determination information receiving unit F34, an additional information transmitting unit F35, a guide information receiving unit F36, a personal information transmitting unit F37, and a reservation information transmitting unit F38.

In this embodiment, each function provided in the mobile terminal 3 is executed by the CPU 31, which is a general-purpose processor. However, some or all of these functions may be performed by one or more dedicated processors, hardware arithmetic circuits, and the like. Here, the hardware arithmetic circuit means, for example, an adder circuit, a multiplying circuit, a flip-flop, or the like in which logic gates are combined. Further, some or all of these functions may be executed by a separate computer.

The measurement data from the Raman spectroscopic device 2 is input to the input unit F31. Further, the transmission unit F32 transmits the measurement data input to the input unit F31 to the server 5. The schedule information receiving unit F33 receives the health management schedule information of the subject, which is determined by a schedule planning unit F54 of the server 5 and transmitted by a schedule information transmitting unit F55. The second determination information receiving unit F34 receives the determination result data which is determined by a determination unit F52 of the server 5 and transmitted by a determination information transmitting unit F53. The additional information transmitting unit F35 transmits additional information regarding the health condition of the subject. The guide information receiving unit F36 receives guide information transmitted by the guide information transmitting unit F74 of the hospital terminal 7. The personal information transmitting unit F37 transmits the personal data of the subject for registration in a personal information database D501 in the server 5. The reservation information transmitting unit F38 transmits reservation information including a desired date and time to the dental clinic selected by the schedule planning unit F54 of the server 5.

### (Functions of Server 5)

The server 5 reads the program stored in the auxiliary storage device 54 into the RAM 52 and executes the program by the CPU 51, and functions as a computer which includes the personal information database D501, an institution information database D502, a determination information database D503, and a measurement information receiving unit F51, the determination unit F52, the determination information transmitting unit F53, the schedule planning unit F54, the schedule information transmitting unit F55, a personal information receiving unit F56, an institution information receiving unit F57, and a reservation information receiving unit F58.

In the personal information database D501, personal data as personal information transmitted and registered from one or more mobile terminals 3 is accumulated. The personal information database D501 corresponds to a personal information storage unit. In the institution information database D502, hospital data as institution information transmitted from one or more hospital terminals 7 is accumulated. The institution information database D502 corresponds to an institution information storage unit. The determination information database D503 stores the relationship between the calculated value obtained by calculating the measurement data and the tooth condition. This relationship corresponds to a determination reference. The determination information database D503 corresponds to a determination reference storage unit. Then, these databases are constructed by the program of a database management system managing the data stored in the auxiliary storage device 54.

The measurement information receiving unit F51 receives the measurement data of Raman spectroscopic measurement transmitted from the transmission unit F32 of the mobile terminal 3. The determination unit F52 refers to the determination information database D503 for the measurement data of the spectroscopic measurement received by the measurement information receiving unit F51, and determines the tooth condition. The determination information transmitting unit F53 transmits the determination result data which is the information of the result determined by the determination unit F52. The schedule planning unit FM refers to the personal data of the subject in the personal information database D501 and the hospital data in the institution information database D502, selects the dental clinic to which the subject should visit for dental health management, and creates schedule information. The schedule information transmitting unit F55 transmits the schedule information created by the schedule planning unit FM. The personal information receiving unit F56 receives the personal data of the subject transmitted from the personal information transmitting unit F37 of the mobile terminal 3. The institution information receiving unit F57 receives the hospital data of the dental clinic transmitted from an institution information transmitting unit F75 of the hospital terminal 7. The reservation information receiving unit F58 receives the reservation information such as the desired date and time of reservation transmitted from the mobile terminal 3.

### (Functions of Hospital Terminal 7)

The hospital terminal 7 reads a program stored in the auxiliary storage device 74 into the RAM 72 and executes the program by the CPU 71, and functions as a computer which includes a determination information receiving unit F71, a second schedule information receiving unit F72, an additional information receiving unit F73, the guide information transmitting unit F74, the institution information transmitting unit F75, and a reservation information receiving unit F76. Further, some or all of these functions included in the hospital terminal 7 may be executed by one or more dedicated processors, hardware arithmetic circuits, and the like.

The determination information receiving unit F71 receives the determination result data which is determined by a determination unit F52 of the server 5 and transmitted by a determination information transmitting unit F53. The second schedule information receiving unit F72 receives the health management schedule information of the subject, which is determined by a schedule planning unit F54 of the server 5 and transmitted by a schedule information transmitting unit F55. The additional information receiving unit F73 receives the additional information regarding the health condition of the subject transmitted from the additional information transmitting unit F35 of the mobile terminal 3. The guide information transmitting unit F74 transmits guide information including a message recommending that the subject visit the hospital, based on the determination information transmitted from the determination information transmitting unit F53 of the server 5. The institution information transmitting unit F75 transmits the hospital data of the dental clinic. The reservation information receiving unit F76 receives the reservation information such as the desired date and time of reservation transmitted from the mobile terminal 3.

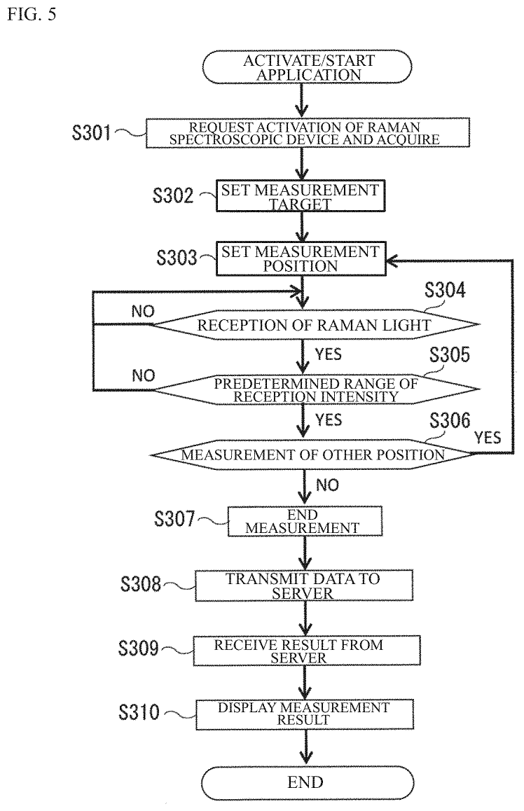

Next, the control in the measurement terminal 4 will be described. FIG. 5 is a flowchart of a measurement routine executed when Raman spectroscopic measurement is performed in this embodiment. This measurement routine is a program that forms a part of the health management application that is downloaded via the Internet and stored in the auxiliary storage device 34 of the mobile terminal 3.

When this routine is executed, first, in Step S301, the state becomes a request state of activation of the Raman spectroscopic device 2, and the mobile terminal 3 displays an input request screen as to whether the Raman spectroscopic device 2 is activated. Then, when the user touches an activation button, the command for activating the Raman spectroscopic device 2 is acquired. As a result, the laser light source 2a is turned on and the Raman spectroscopic device 2 is ready for measurement. When the process of Step S301 ends, the process proceeds to Step S302.

In Step S302, setting of the measurement purpose is required. More specifically, the mobile terminal 3 displays options for measurement purpose and a screen requesting selection of any of the options. Then, the measurement purpose is set by the user selecting an appropriate purpose. When the process of Step S302 ends, the process proceeds to Step S303.

In Step S303, the measurement position is set. More specifically, which part of which tooth is to be measured is selected. When the process of Step S303 ends, the process proceeds to Step S304.

In Step S304, the excitation light of the Raman spectroscopic device 2 is actually irradiated to the location designated in Step S303, and the Raman scattered light is received. The reception of the Raman scattered light is detected when the output signal from the photodetector 2g of the Raman spectroscopic device 2 increases above a threshold. If it is not determined that the Raman scattered light is received here, the process returns to before this process, and this process is repeated until it is determined that the Raman scattered light is received. If it is determined that Raman scattered light has been received, the process proceeds to Step S305.

In Step S305, it is determined whether the received light intensity of the Raman scattered light detected by the photodetector 2g is within a predetermined range. Here, the excitation light of the Raman spectroscopic device 2 is irradiated, but when the Raman scattered light of sufficient intensity for measurement is not obtained due to the relationship of irradiation angle and distance, the irradiation angle and the distance may be changed and adjusted so that a sufficient received light intensity can be obtained. If it is determined that the received light intensity in the predetermined range has not been obtained, the process returns to the process before Step S304, and the processes of Steps S304 and S305 are repeatedly executed until it is determined in Step S305 that the received light intensity in the predetermined range is obtained. When the received light intensity within the predetermined range is obtained in Step S305, the process proceeds to Step S306.

In Step S306, it is determined whether to continue measurement at another position. Specifically, a screen prompting the user to select whether to measure another position is displayed on the mobile terminal 3. Then, when the user selects YES or NO, it is determined whether to continue the measurement at another position. If the user selects YES here, the process returns to Step S303. On the other hand, if the user selects NO, the process proceeds to Step S307.

The measurement ends in Step S307. More specifically, the power of the Raman spectroscopic device 2 is turned off, and the screen of the mobile terminal 3 displays a screen indicating that the measurement is completed. When the process of Step S307 ends, the process proceeds to Step S308.

In Step S308, the measurement data as the measurement result is transmitted to the server 5. When the process of Step S308 ends, the process proceeds to Step S309.

In Step S309, the determination result data as the determination information is transmitted from the server 5 and received by the mobile terminal 3. When the reception is completed, the process proceeds to Step S310. In Step S310, the tooth condition is displayed on the mobile terminal 3. When the process of Step S310 ends, this routine ends once. In the above measurement routine, the processes of Steps S301 to S307 correspond to the spectroscopic measurement step in this embodiment. The process of Step S308 corresponds to the measurement result transmitting step.

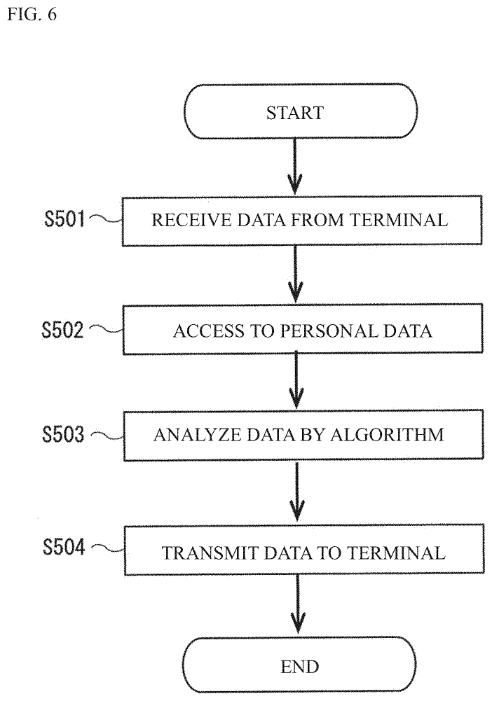

Next, a determination process in the server 5 will be described. FIG. 6 illustrates a flowchart of the determination routine in this embodiment.

When this routine is executed, first, in Step S501, the measurement data from the mobile terminal 3 is received. When the process of Step S501 ends, the process proceeds to Step S502. In Step S502, the subject is identified by accessing the personal information database D501. When the process of Step S502 ends, the process proceeds to Step S503. In Step S503, the measurement data is analyzed by the determination algorithm. The details of this analysis method will be described later. The tooth condition is analyzed by the analysis in Step S503. Here, the measurement data and the determination result data are stored together with the date in association with the personal data in the personal information database D501. When the process of Step S503 ends, the process proceeds to Step S504.

In Step S504, the determination result data is transmitted to the mobile terminal 3. When the process of Step S504 ends, this routine ends once. In the above determination routine, the process of Step S503 corresponds to the determination step. The process of Step S504 corresponds to a determination information transmitting step.

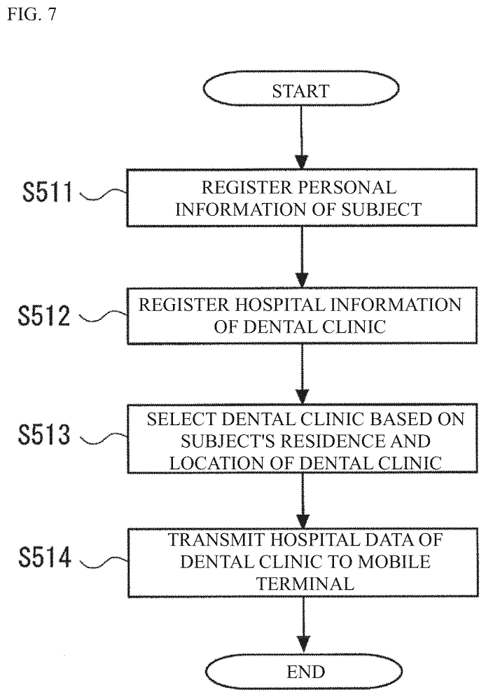

Next, a schedule planning process in the server 5 will be described. FIG. 7 illustrates a flowchart of the schedule planning routine in this embodiment.

When this routine is executed, the personal data of the subject is registered in the server 5 in Step S511. This may be performed by providing a registration function in the health management application downloaded to the mobile terminal 3, the user selecting execution of this registration function, inputting the personal data of the subject to the mobile terminal 3, and instructing registration. Examples of this personal data include name, age, sex, residence, contact information, medical history, and the like. As the contact information, a telephone number, a fax number, and an electronic mail address can be exemplified. The personal data transmitted here is stored in the auxiliary storage device 54 as data of the personal information database D501 of the server 5. When the process of Step S511 ends, the process proceeds to Step S512.

In Step S512, the hospital data of the dental clinic is registered in the server 5. This is performed by causing the hospital terminal 7 to execute the registration function, inputting hospital data of the dental clinic to the hospital terminal 7, and instructing registration. Examples of this hospital data include name, location, specialty field, possessed equipment, contact information, etc. of the hospital. As the contact information, a telephone number, a fax number, and an electronic mail address can be exemplified. The hospital data transmitted here is stored in the auxiliary storage device 54 as data of the institution information database D502 of the server 5. When the process of Step S512 ends, the process proceeds to Step S513.

In Step S513, the dental clinic to visit is selected from the subject's residence in the personal information database D501 and the location information of the dental clinic in the institution information database D502. More specifically, a dental clinic in the same prefecture as the subject's residence is primarily selected. If this does not limit the number to an appropriate number, the range is narrowed down to the same city as the subject's residence, the same town as the subject's residence, and so on. This number may be plural or may be one. When the process of Step S513 ends, the process proceeds to Step S514.

In Step S514, the hospital data of the selected dental clinic is transmitted to the mobile terminal 3. When the processing of S514 ends, this routine ends once. In the above schedule planning routine, the process of Step S511 corresponds to a personal information transmitting step and a personal information storing step. The process of Step S512 corresponds to an institution information transmitting step and an institution information storing step. The process of Step S513 corresponds to a schedule planning step. Further, the process of Step S514 corresponds to a schedule information transmitting step. The hospital data of the selected dental clinic corresponds to the schedule information.

In the above schedule planning process, a dental clinic to visit is selected based on a location value of the dental clinic. However, the selection reference of the dental clinic is not limited to this. For example, the dental clinic may be selected with reference to the determination result data of the Raman spectroscopic measurement, based on the specialty field required according to the determination result or the possessed equipment. Alternatively, these references may be combined and selected.

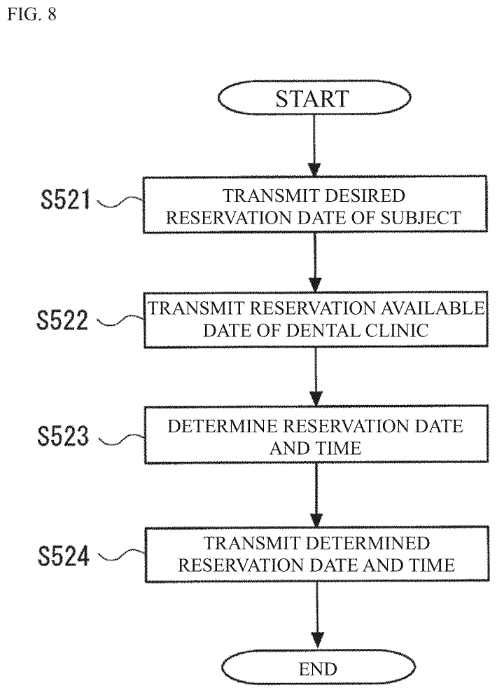

Next, after the dental clinic to visit is selected, the selected dental clinic may be reserved and reception support may be provided. FIG. 8 illustrates a flowchart of the reservation acceptance support routine executed in this case. When this routine is executed, first, in Step S521, the desired reservation date of the subject is transmitted as reservation information. More specifically, on the mobile terminal 3, a screen requesting input of a desired reservation date and time is displayed while the health management application is being executed. At that time, prioritization may be requested such as desired date and time 1, 2, and 3. When the user presses a send button after inputting the desired reservation date and time, the information on the desired reservation date and time is transmitted from the mobile terminal 3 to the server 5. When the process of Step S521 ends, the process proceeds to Step S522.

In Step S522, the information on the reservation available date and time of the dental clinic is transmitted from the hospital terminal 7 to the server 5. When the process of Step S522 ends, the process proceeds to Step S523.

In Step S523, the server 5 determines the reservation date and time. More specifically, with respect to the desired reservation date and time information transmitted from the mobile terminal 3 and received at the hospital terminal 7, the reservation available date and time transmitted from the hospital terminal 7 is referred in descending order of priority. Then, the date and time that first matches the reservation available date and time is determined as the reservation date and time among the desired reservation dates and times. When the process of Step S523 ends, the process proceeds to Step S524. In Step S524, the information on the reservation date and time is transmitted from the server 5 to the mobile terminal 3 and the hospital terminal 7. When the process of Step S524 ends, this routine ends once. In the above reservation acceptance support routine, the process of Step S523 corresponds to the schedule planning step. Further, the process of Step S524 corresponds to the schedule information transmitting step.

As a result, in order to manage the tooth condition of the subject, not only a dental clinic is selected, but also reservation/reception can be completed promptly. Therefore, it is possible to more efficiently maintain and improve the health condition of the subject. In the above reservation acceptance support routine, the desired reservation date and time is sent from the mobile terminal 3 to the server 5 and the hospital terminal 7, the reservation date and time is determined by the hospital terminal 7, and the determined reservation date and time may be transmitted from the hospital terminal 7 to the server 5 and the mobile terminal 3. Alternatively, the reservation available date and time is sent from the hospital terminal 7 to the server 5 and the mobile terminal 3, the reservation date and time is determined by the mobile terminal 3, and the determined reservation date and time may be transmitted from the mobile terminal 3 to the server 5 and the hospital terminal 7.

After that, when the subject actually visits the dental clinic, the information may be transmitted from the mobile terminal 3 or the hospital terminal 7 to the server 5. The server 5 stores a visit history in the auxiliary storage device 54 in association with the personal information database D501 and the institution information database D502. As a result, the server 5 can manage the scheduled progress, that is, the visit information.

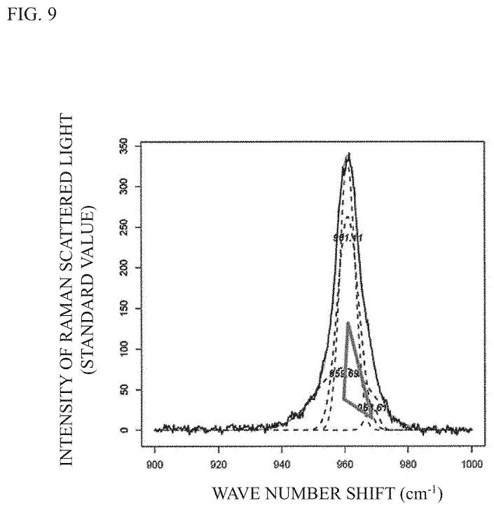

Next, the outline of the determination algorithm for determining the tooth condition from the measurement data of Raman scattered light will be described. As described above, the Raman scattered light has a wave number shift corresponding to the molecular structure of the subject with respect to the excitation light. FIG. 9 illustrates an example of a spectroscopic measurement result of Raman scattered light when the tooth surface is irradiated with excitation light. The horizontal axis represents the wave number shift, and the vertical axis represents the received light intensity of the standardized Raman scattered light.

In this embodiment, a dental caries state is determined using a condition coefficient Qt represented by the following Expression (1).

$$\begin{matrix}[ Math . 1 ] & \\ {Q}_{t} = \frac{{W}_{961}}{{W}_{r}} \frac{α {I}_{970} + β {I}_{957}}{{I}_{970} + {I}_{957} + {I}_{961}} & ( 1 )\end{matrix}$$

Here, Qt is a particular tooth condition coefficient, I is the integrated area intensity of a peak at a particular wave number shift, W is the width of the peak at a particular wave number shift, and Wr is the width of a reference peak for pure hydroxyapatite or complete enamel. α and β are coefficients of 0 or more and 1 or less, which correct the relative intensities of the subbands.

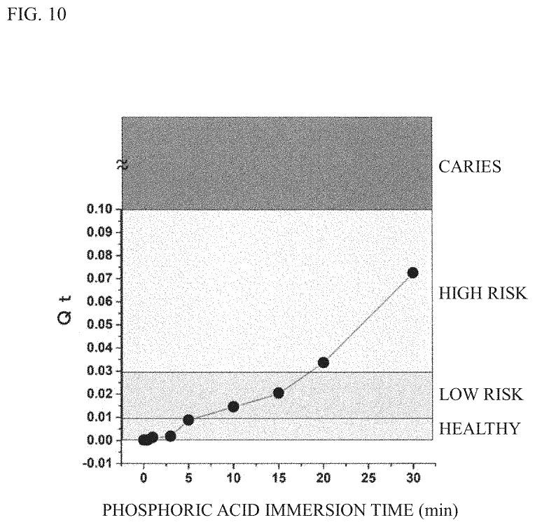

FIG. 10 illustrates the relationship between the tooth condition and the condition coefficient Qt. The horizontal axis of FIG. 10 is time (min) when the tooth is immersed in phosphoric acid, and the vertical axis is the value of the condition coefficient Qt. Here, α=1, β=0, and Wr=9.5. As can be seen from FIG. 10, the value of the condition coefficient Qt increases as the phosphoric acid immersion time increases and the tooth surface becomes more damaged. In this embodiment, from this graph, when the value of the condition coefficient Qt is 0.01 or less, it is healthy. When the value of the condition coefficient Qt is 0.01 or more and 0.03 or less, the risk is low. When the value of the condition coefficient Qt is in a range of 0.03 or more and 0.10 or less, the risk is high. When the condition coefficient Qt is 0.1 or more, it is determined to be caries (cavities).

In this embodiment, the table of the relationship between the value of the condition coefficient Qt and the tooth condition as illustrated in FIG. 10 is stored in the auxiliary storage device 54 of the server 5 as the determination information database D503. This process corresponds to a determination reference forming step in this embodiment. Then, the value of the condition coefficient Qt is calculated from the measurement data of the Raman scattered light received from the mobile terminal 3, the tooth condition is derived from the determination information database D503, and the result is transmitted to the mobile terminal 3 as determination result data. Therefore, it is possible to determine and manage the tooth health condition from the stage before the tooth is in the caries state.

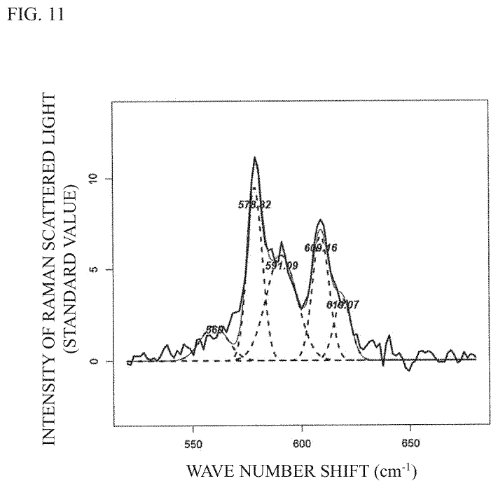

Incidentally, the above determination algorithm, as illustrated in Equation (2) and FIG. 11, a region of about 600 cm⁻¹, particularly a ratio between the strongest peak of hydroxyapatite appearing at about 580 cm⁻¹ and the shoulders associated with tricalcium phosphate appearing at about 560 cm⁻¹ is taken into account for the wave number shift, so accuracy can be achieved.

$$\begin{matrix}[ Math . 2 ] & \\ {Q}_{t} = \frac{{W}_{961}}{{W}_{r}} \frac{α {I}_{970} + β {I}_{957}}{{I}_{970} + {I}_{957} + {I}_{961}} + γ \frac{{I}_{560}}{{I}_{580}} & ( 2 )\end{matrix}$$

As described above, according to this embodiment, the subject can easily know the dental health condition at home. Further, it is possible to obtain information on the dental clinic to visit based on the personal data such as the subject's residence and the determination result data. Further, the reservation process can quickly proceed with the dental clinic to visit, and the tooth condition can be efficiently managed.

In addition, in the above embodiment, an example in which the invention is applied to the management of the tooth condition has been described. However, the subject of health management to which the invention is applied is not limited to the tooth condition. Hereinafter, another example of health management using the measurement of Raman scattered light will be described.

### (Management of Skin Condition)

When managing the skin condition by measuring the Raman scattered light, the following Expression (3) is used, for example.

$$\begin{matrix}[ Math . 3 ] & \\ {R}_{PF} = \frac{{I}_{d}}{{I}_{α}} = {k}_{1} {e}^{( - {k}_{2} {x}_{A} )} & ( 3 )\end{matrix}$$

Here, R_{PF} is the intensity ratio of I_{d} and I_{α}. I_{d} is the integrated area intensity of a peak in the band with the wave number (cm⁻¹) of 1681 to 1682 and corresponds to the C═O stretching motion in the disordered structure (amide I). In addition, I_{α} is the integrated area intensity of a peak in the band of wave numbers (cm⁻¹) of 1652 to 1654, and corresponds to the C═O stretching motion in α-helix (amide I). Then, k₁ and k₂ are constants that depend on test conditions, laser light, and characteristics of the Raman spectroscopic device 2.

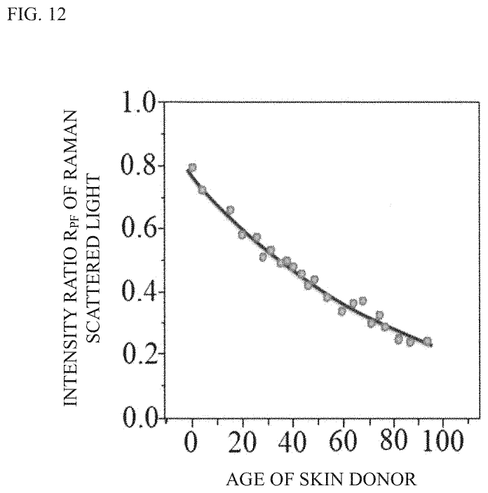

FIG. 12 illustrates a graph of the relationship between the age of the skin donor and the intensity ratio R_{PF}. In this drawing, k₁=0.77 and k₂=0.012. Thus, there is a clear correlation between the intensity ratio R_{PF} and skin age. Therefore, in the auxiliary storage device 54 of the server 5, the determination information database D503 indicating the relationship between age and R_{PF} is stored, the measurement data of Raman spectroscopic measurement is received from the mobile terminal 3, the skin age is determined, and the determination result data may be transmitted to the mobile terminal 3.

$$\begin{matrix}[ Math . 4 ] & \\ {R}_{L C 1} = \frac{{I}_{h + o + cp}}{{I}_{1} + {I}_{5}} = BMI * {k}_{1} * {e}^{( - {k}_{2} {x}_{A}^{{k}_{3}} )} & ( 4 )\end{matrix}$$

It is also possible to manage the BMI and obesity status from the skin condition. In that case, the following Expressions (4) and (5) may be used.

$$\begin{matrix}[ Math . 5 ] & \\ {R}_{LC 2} = \frac{{I}_{h + o}}{{I}_{l}} = BMI * {k}_{1} * {e}^{( - {k}_{2} {x}_{A}^{{k}_{3}} )} & ( 5 )\end{matrix}$$

Here, R_{LC1} is the intensity ratio of (I_{h+o+cp}) and (I₁+I₅). R_{LC2} is the intensity ratio of (Iₕ₊ₒ) and Iₗ. Iₕ is the integrated area intensity of a peak in the band of wave numbers (cm⁻¹) of 2877 to 2879 and corresponds to vs (CH₃) in a hexagonal lipid. Further, Iₒ is the integrated area intensity of a peak in the band of wave numbers (cm⁻¹) of 2882 to 2888, and corresponds to vs (CH₃) in the orthorhombic lipid. I_{cp} is the integrated area intensity of a peak in the band of wave numbers (cm⁻¹) of 2902 to 2910, and corresponds to vs (CH₂) and vs (CH₃) in cholesterol and phospholipid. k₁, k₂, and k₃ are constants that depend on experimental conditions, and BMI (Body Mass Index) is a body mass index that differs between R_{LC1} and R_{LC2}.

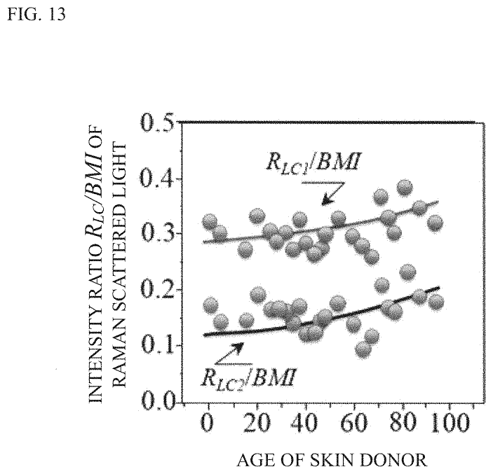

FIG. 13 is a diagram illustrating an example of the relationship between the intensity ratio R_{LC}/BMI and the age of the skin donor. From this drawing, it can be seen that there is a strong correlation between R_{LC} and the BMI and the age of the skin donor. Therefore, the relationship between age, R_{LC1}, R_{LC2}, and the BMI is stored in the auxiliary storage device 54 of the server 5 as the determination information database D503, and the measurement data of Raman spectroscopy results and the age data are received from the mobile terminal 3. The BMI and the degree of obesity can be determined, and it is also possible to determine the skin age by receiving the measurement data of the Raman spectroscopy result and the data of the BMI and the degree of obesity from the mobile terminal 3.

### (Management of Bone Condition)

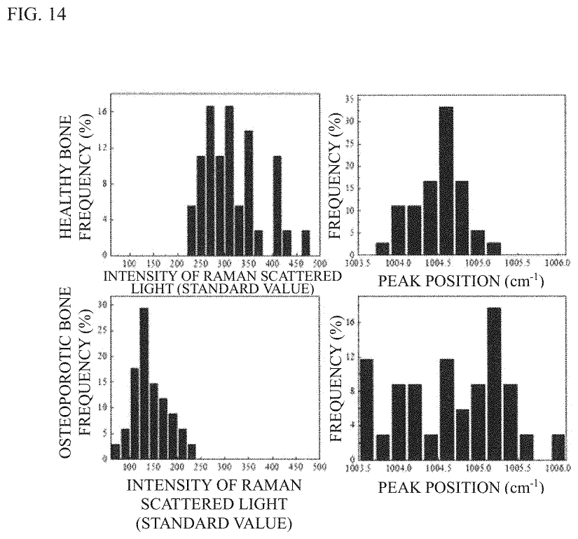

FIG. 14 illustrates the statistically summarized results obtained by measuring the phenylalanine peak of a healthy bone and an osteoporotic bone at a wave number (cm⁻¹) near 1005 by Raman scattering spectroscopy. As can be seen from FIG. 14, in the bone with osteoporosis, the wave number of the phenylalanine peak varies and a Raman scattered light intensity is lower than that in the healthy bone.

Therefore, when managing the bone condition by measuring the Raman scattered light, the following Expressions (6) to (8) may be used, for example.

$$\begin{matrix}[ Math . 6 ] & \\ {P}_{c}^{r} = \frac{{I}_{1005}}{α {I}_{1652} + β {I}_{1444}} & ( 6 ) \\ [ Math . 7 ] & \\ {P}_{b}^{r} = \frac{{I}_{1005}}{α {I}_{960} + β {I}_{971}} & ( 7 ) \\ [ Math . 8 ] & \\ {Q}_{c}^{r} = \frac{{I}_{1444}}{{I}_{1652}} & ( 8 )\end{matrix}$$

Here, I₁₀₀₅ is a phenylalanine peak. Phenylalanine has a wide range of known metabolic effects and is also associated with mineral deficiency in bone. The intensity ratios of Expressions (6) to (8) can be used to manage the amount of collagen and minerals in bone.

Therefore, the server 5 stores the relationship between the amounts of collagen and minerals, or the risk of osteoporosis and P_{c}^{r}, P_{b}^{r}, and Q_{c}^{r} as the determination information database D503, and receives the measurement data of Raman spectroscopic measurement from the mobile terminal 3. It is also possible to determine the amount of collagen and minerals or the risk of osteoporosis.

The bone condition can be evaluated as a combination of bone mineral content and at least two main components of the biomatrix. Since the Raman spectrum of bone has many regions and peaks, it is possible to evaluate the bone condition by various methods other than the above by Raman spectroscopic measurement.

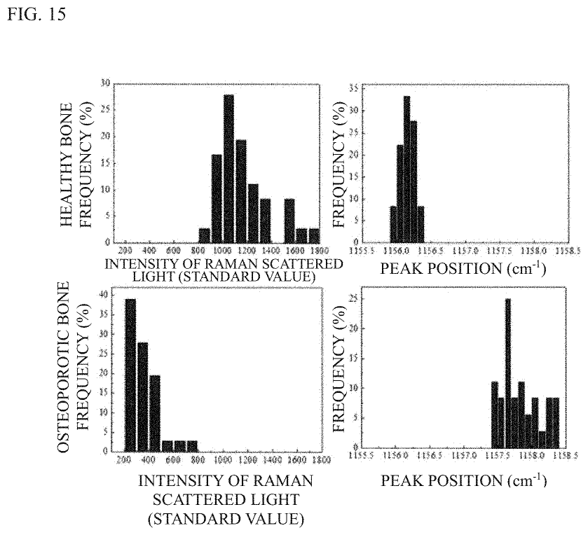

For example, the mineral content of bone can be determined by the same method as the determination of tooth enamel because it is composed of hydroxyapatite. Alternatively, it is possible to determine the state of osteoporosis or osteonecrosis from the decrease in the intensity of Raman peaks associated with sphingomyelin, Raman peaks associated with phospholipids, and Raman peaks associated with lipid loading and viscosity. FIG. 15 illustrates the statistically summarized results of Raman scattering spectroscopy for Raman peaks related to sphingomyelin in healthy bones and osteoporotic bones. As can be seen from FIG. 15, in the osteoporotic bone, the wave number of the sphingomyelin peak is increased and the Raman scattered light intensity is lowered than that in the healthy bone.

### REFERENCE SIGNS LIST

1 health management system
2 Raman spectroscopic device
3 mobile terminal
4 measurement terminal
5 server
7 hospital terminal

1. A health condition management system, comprising:
a spectroscopic measurement device that irradiates a part of a subject's body with light and detects a scattered light or a reflected light from the subject's body to perform spectroscopic measurement;
a subject terminal to which an output signal of the spectroscopic measurement device is input;
an information processing device that is capable of communicating with the subject terminal; and
a management institution terminal that is provided in a management institution for managing a health condition of the subject and capable of communicating with the subject terminal and the information processing device,
wherein the subject terminal includes
an input unit to which a measurement result by the spectroscopic measurement device is input, and
a transmission unit that transmits the measurement result input to the input unit to the information processing device,
the information processing device includes
a measurement information receiving unit that receives the measurement result transmitted from the transmission unit of the subject terminal,
a determination unit that determines a health condition of the subject based on the measurement result received by the measurement information receiving unit and a predetermined algorithm, and
a determination information transmitting unit that transmits determination information that is information of a result determined by the determination unit,
the management institution terminal includes a determination information receiving unit that receives the determination information transmitted by the determination information transmitting unit of the information processing device,
the information processing device further includes
a schedule planning unit that determines a management plan of a health condition of the subject based on the determination information, and
a schedule information transmitting unit that transmits a schedule information that is information of a management schedule of a health condition of the subject determined by the schedule planning unit to the subject terminal and/or the management institution terminal,
the subject terminal includes
a second determination information receiving unit that receives the determination information transmitted by the determination information transmitting unit of the information processing device, and
a schedule information receiving unit that receives the schedule information transmitted by the schedule information transmitting unit of the information processing device, and
the management institution terminal includes a second schedule information receiving unit that receives the schedule information transmitted by the schedule information transmitting unit of the information processing device.

2. The health condition management system according to claim 1, wherein
the subject terminal includes an additional information transmitting unit that transmits additional information regarding a health condition of the subject to the management institution terminal, and
the management institution terminal further includes an additional information receiving unit that receives the additional information transmitted from the additional information transmitting unit of the subject terminal.

3. The health condition management system according to claim 1, wherein
the management institution terminal further includes a guide information transmitting unit that transmits guide information for guiding the subject to be managed by the management institution based on the determination information, and
the subject terminal further includes a guide information receiving unit that receives the guide information transmitted by the guide information transmitting unit.

4. The health condition management system according to claim 1, wherein
the subject terminal further includes a personal information transmitting unit that transmits personal information of the subject to the information processing device,
the management institution terminal further includes an institution information transmitting unit that transmits institution information of the management institution to the information processing device,
the information processing device further includes
a personal information receiving unit that receives the personal information,
an institution information receiving unit that receives the institution information,
a personal information storage unit that stores one or more pieces of the personal information, and
an institution information storage unit that stores one or more pieces of the institution information, and
the schedule planning unit determines a management schedule of the health condition of the subject based on the personal information of the subject stored in the personal information storage unit and the institution information of the management institution stored in the institution information storage unit.

5. The health condition management system according to claim 4, wherein
the institution information includes information on name, location, and contact information of the management institution,
the personal information includes information about the subject's residence,
the schedule planning unit selects a management institution whose residence is closer to the subject's residence, and
the schedule information includes at least one of name, address, and contact information of the selected management institution.

6. The health condition management system according to claim 1, wherein
the subject terminal further includes a reservation information transmitting unit that transmits, to the information processing device and/or the management institution terminal, reservation information including a desired reservation date, which is included in the schedule information and desired to be reserved in the management institution selected by the schedule planning unit, and
the information processing device and/or the management institution terminal further includes a reservation information receiving unit that receives the reservation information.

7. The health condition management system according to claim 1, wherein the spectroscopic measurement device is a Raman spectroscopic device that spectroscopically measures Raman scattered light from the subject using a Raman spectroscopic technique.

8. The health condition management system according to claim 7, wherein
the information processing device includes a determination reference storage unit that stores a relationship between a calculated value obtained by adding a predetermined calculation to at least one of a Raman scattered light intensity at a predetermined frequency, a peak width, and a peak position and a health condition of the subject, and
the determination unit refers the measurement result received by the measurement information receiving unit to the relationship stored in the determination reference storage unit, and determines the health condition of the subject based on an algorithm for deriving the health condition of the subject which corresponds to the measurement result.

9. The health condition management system according to claim 1, wherein
a part of the subject's body is a subject's teeth,
the health condition is a tooth condition, and
the management institution is a dental clinic.

10.-19. (canceled)

20. The health condition management system according to claim 2, wherein
the management institution terminal further includes a guide information transmitting unit that transmits guide information for guiding the subject to be managed by the management institution based on the determination information, and
the subject terminal further includes a guide information receiving unit that receives the guide information transmitted by the guide information transmitting unit.

21. The health condition management system according to claim 2, wherein
the subject terminal further includes a personal information transmitting unit that transmits personal information of the subject to the information processing device,
the management institution terminal further includes an institution information transmitting unit that transmits institution information of the management institution to the information processing device,
the information processing device further includes
a personal information receiving unit that receives the personal information,
an institution information receiving unit that receives the institution information,
a personal information storage unit that stores one or more pieces of the personal information, and
an institution information storage unit that stores one or more pieces of the institution information, and
the schedule planning unit determines a management schedule of the health condition of the subject based on the personal information of the subject stored in the personal information storage unit and the institution information of the management institution stored in the institution information storage unit.

22. The health condition management system according to claim 21, wherein
the institution information includes information on name, location, and contact information of the management institution,
the personal information includes information about the subject's residence,
the schedule planning unit selects a management institution whose residence is closer to the subject's residence, and
the schedule information includes at least one of name, address, and contact information of the selected management institution.

23. The health condition management system according to claim 2, wherein
the subject terminal further includes a reservation information transmitting unit that transmits, to the information processing device and/or the management institution terminal, reservation information including a desired reservation date, which is included in the schedule information and desired to be reserved in the management institution selected by the schedule planning unit, and
the information processing device and/or the management institution terminal further includes a reservation information receiving unit that receives the reservation information.

24. The health condition management system according to claim 2, wherein the spectroscopic measurement device is a Raman spectroscopic device that spectroscopically measures Raman scattered light from the subject using a Raman spectroscopic technique.

25. The health condition management system according to claim 24, wherein
the information processing device includes a determination reference storage unit that stores a relationship between a calculated value obtained by adding a predetermined calculation to at least one of a Raman scattered light intensity at a predetermined frequency, a peak width, and a peak position and a health condition of the subject, and
the determination unit refers the measurement result received by the measurement information receiving unit to the relationship stored in the determination reference storage unit, and determines the health condition of the subject based on an algorithm for deriving the health condition of the subject which corresponds to the measurement result.

26. The health condition management system according to claim 2, wherein
a part of the subject's body is a subject's teeth,
the health condition is a tooth condition, and
the management institution is a dental clinic.
